(19) United States
(12) Patent Application Publication
(10) Pub. No.: US 2026/0076812 A1
(43) Pub. Date: Mar. 19, 2026

(54) ORTHOTIC AND PROSTHETIC DEVICE AND MANUFACTURING SYSTEM AND METHOD

(71) Applicant: Hanger, Inc., Austin, TX (US)

(72) Inventors: Aaron Flores, Austin, TX (US); Antonio Dias, Scottsdale, AZ (US); Justin Mieth, Austin, TX (US); Michael E. Tompkins, Austin, TX (US)

(73) Assignee: Hanger, Inc., Austin, TX (US)

(21) Appl. No.: 19/399,481

(22) Filed: Nov. 24, 2025

Related U.S. Application Data
(63) Continuation-in-part of application No. 18/594,940, filed on Mar. 4, 2024.
Application No. 18/594,940 is a continuation of application No. 17/373,147, filed on Jul. 12, 2021, now Pat. No. 12,150,870.
Application No. 17/373,147 is a continuation-in-part of application No. 16/050,849, filed on Jul. 31, 2018, now Pat. No. 11,058,557.
Application No. 16/050,849 is a continuation of application No. PCT/US2017/015981, filed on Feb. 1, 2017.
(63) Continuation-in-part of application No. 17/547,500, filed on Dec. 10, 2021.
(60) Provisional application No. 62/290,254, filed on Feb. 2, 2016.
(60) Provisional application No. 63/124,230, filed on Dec. 11, 2020.

(51) Int. Cl. A61F 2/80; A61F 2/78; B33Y 50/00; B33Y 80/00

(52) U.S. Cl. CPC A61F 2/80; B33Y 80/00; A61F 2002/7875; A61F 2002/802; B33Y 50/00

(57) ABSTRACT

A prosthetic device includes a socket, fasteners, and a pylon. The socket defines a cavity configured to receive a residual limb of a user. The socket includes a base defining multiple blind-holes. Each of the fasteners are configured to be received within a corresponding one of the blind-holes. The fasteners each include internal threads. The pylon includes through-holes that are aligned with a corresponding one of the blind-holes. The pylon is configured to be directly coupled with the base of the socket through externally threaded fasteners that extend through the through-holes and threadingly couple with the internal threads of the plurality of fasteners.

### CROSS-REFERENCE TO RELATED PATENT APPLICATIONS

This application: (i) is a continuation-in-part of U.S. application Ser. No. 18/594,940, filed Mar. 4, 2024, which is a continuation of U.S. application Ser. No. 17/373,147, filed Jul. 12, 2021, which is a continuation-in-part of U.S. application Ser. No. 16/050,849, filed Jul. 31, 2018, which is a continuation of International Application No. PCT/US2017/015981, filed Feb. 1, 2017, which claims priority to U.S. Provisional Patent Application No. 62/290,254, filed on Feb. 2, 2016; and (ii) is a continuation-in-part of U.S. patent application Ser. No. 17/547,500, filed Dec. 10, 2021, which claims priority to U.S. Provisional Application No. 63/124,230, filed Dec. 11, 2020, the entire disclosure of which is incorporated by reference herein.

### BACKGROUND

The present disclosure relates generally to prosthetics and orthotics. More particularly, the present disclosure relates to additive manufacturing or protective devices, prosthetics and/or orthotics.

### SUMMARY OF THE INVENTION

Various example embodiments relate to a prosthetic device including a socket, a pylon, and a connection insert. In some embodiments, the socket defines a cavity configured to receive a residual limb of a user. In some embodiments, the pylon is coupled to a distal end of the socket by a threaded fastener. In some embodiments, the connection insert is within the socket and is configured to receive the threaded fastener to couple the pylon with the socket. In some embodiments, at least one of a vacuum line or a wire passes from an exterior of the socket and through an opening of the connection insert to provide fluid communication between the exterior and the cavity.

In some embodiments, the socket is formed by a three-dimensional printing process. In some embodiments, the connection insert includes an internally threaded fastener aligned with a through hole in the socket, wherein the internally threaded fastener is structured to receive the threaded fastener thereby securing the pylon to the socket.

In some embodiments, the connection insert is made of metal. In some embodiments, the socket is made of three-dimensionally printed plastic. In some embodiments, the connection insert defines multiple internally threaded fasteners disposed about the connection insert at corners of a square pattern.

In some embodiments, the connection insert is X-shaped. In some embodiments, the connection insert is U-shaped.

Another embodiment relates to a prosthetic device including a socket, a pylon, and a connection insert. In some embodiments, the socket defines a cavity configured to receive a residual limb of a user. In some embodiments, the pylon is coupled to a distal end of the socket by a plurality of threaded fasteners. In some embodiments, the connection insert within the socket includes multiple internally threaded fasteners configured to receive the plurality of threaded fasteners to couple the pylon with the socket. In some embodiments, the socket extends along an entirety of a top surface of the connection insert.

In some embodiments, the socket is formed by a three-dimensional printing process. In some embodiments, the connection insert includes multiple arms and multiple outer rings. In some embodiments, each of the multiple internally threaded fasteners is positioned at one of the outer rings.

In some embodiments, each of the internally threaded fasteners are aligned with a corresponding through hole in the socket. In some embodiments, the internally threaded fasteners are structured to receive the plurality of threaded fasteners thereby securing the pylon to the socket.

In some embodiments, the threaded fasteners include a first internally threaded fastener, a second internally threaded fastener, a third internally threaded fastener, and a fourth internally threaded fastener

In some embodiments, each of the first, second, third, and fourth internally threaded fasteners define a circular opening having a center point. In some embodiments, the first, second, third, and fourth internally threaded fasteners are positioned such that the four center points define vertices of a square.

In some embodiments, the connection insert has an overall X-shape. In some embodiments, the connection insert has an overall U-shape.

Another embodiment relates to a prosthetic device including a socket, multiple fasteners, and a pylon. In some embodiments, the socket defines a cavity configured to receive a residual limb of a user. In some embodiments, the socket includes a base defining multiple blind-holes. In some embodiments, each of the fasteners is configured to be received within a corresponding one of the multiple blind-holes. In some embodiments, the fasteners each include internal threads. In some embodiments, the pylon includes multiple through-holes. In some embodiments, each of the through-holes are aligned with a corresponding one of the blind-holes. In some embodiments, the pylon is configured to be directly coupled with the base of the socket through externally threaded fasteners extending through the through-holes and threadingly coupling with the internal threads of the fasteners.

In some embodiments, the pylon is configured to directly contact an exterior surface of the bottom of the socket when the pylon is directly coupled with the base of the socket. In some embodiments, the fasteners each include the internal threads and external threads. In some embodiments, each of the fasteners are threaded into a corresponding one of the blind-holes.

In some embodiments, the fasteners, the through-holes, the blind-holes, and the externally threaded fasteners include four fasteners, four through-holes, four blind-holes, and four externally threaded fasteners. In some embodiments, the base further includes a center hole extending through an entire thickness of the base. In some embodiments, the blind holes are radially disposed about the center hole.

One implementation of the present disclosure is a method for manufacturing a lower extremity prosthetic socket, according to some embodiments. In some embodiments, the method includes using a digital scanner to capture either an anatomical structure of a patient's distal limb or an anatomical structure of a cast of the patient's distal limb to generate a scan file, converting the scan file to a design file, modifying the design file, and additively manufacturing the design file to produce the lower extremity prosthetic socket using an additive manufacturing device such that the lower extremity prosthetic socket has variable thickness targeting areas of expected stress.

In some embodiments, modifying the design file includes using build-ups or reductions to a thickness of a shell of the design file. In some embodiments, the lower extremity prosthetic socket includes variable thickness along a dimension of the lower extremity prosthetic socket.

In some embodiments, the variable thickness is configured to accommodate the anatomy of the residual limb of the patient. In some embodiments, the variable thickness targets areas of high stress and distributes stress throughout the lower extremity prosthetic socket. In some embodiments, the method further includes identifying areas of high stress based on patient data and modifying the design file such that the lower extremity prosthetic socket has variable thickness targeting the areas of high stress and such that the lower extremity prosthetic socket distributes stress throughout the lower extremity prosthetic socket. In some embodiments, the lower extremity prosthetic socket is configured to distribute forces.

In some embodiments, the method further includes modifying the design file and additively manufacturing the design file such that the lower extremity prosthetic socket comprises trimlines at a proximate end, the trimlines being tapered and having thickness less than the thickness of the lower extremity prosthetic socket proximate a distal end such that the lower extremity prosthetic socket conforms to the anatomical structure of the patient's distal limb. In some embodiments, the method further includes modifying the design file and additively manufacturing the design file such that the lower extremity prosthetic socket is configured to directly couple, at an outer surface, with a pylon at a distal end.

In some embodiments, the method further includes plastically or elastically deforming a part of the lower extremity prosthetic socket after producing the lower extremity prosthetic socket, wherein the part of the lower extremity prosthetic socket is configured to undergo deformation without sustaining structural damage. In some embodiments, the method further includes comprising modifying the design file and additively manufacturing the design file such that an amount of the deformation or flexion at a particular position along the lower extremity prosthetic socket is inversely proportional to a thickness at the particular position along the lower extremity prosthetic socket.

In some embodiments, the design file is at least one of a computer assisted design (CAD) file or a computer assisted manufacturing (CAM) file. In some embodiments, the additive manufacturing device is a 3d printer configured to output layers of material on top of each other in succession to produce the lower extremity prosthetic socket. In some embodiments, the method further includes uploading the design file to the additive manufacturing device.

Another implementation of the present disclosure is a method, according to some embodiments. In some embodiments, the method includes obtaining scan data of a patient's distal limb, using computer assisted design (CAD) or computer assisted manufacturing (CAM) to generate a design file of a lower extremity prosthetic socket based on the scan data, and performing additive manufacturing by a 3D printer to produce the lower extremity prosthetic socket using the design file of the lower extremity prosthetic socket. The lower extremity prosthetic socket has variable thickness determined based on expected stress throughout the lower extremity prosthetic socket.

In some embodiments, the lower extremity prosthetic socket includes a shell having variable thickness along a longitudinal length of the shell and configured to directly couple, at an outer surface, with a pylon at a distal end. In some embodiments, the method further includes generating the design file and performing the additive manufacturing such that the shell comprises trimlines at a proximate end of the lower extremity prosthetic socket, the trimlines being tapered and having thickness less than the thickness of the shell proximate the distal end. In some embodiments, the method further includes generating the design file and performing the additive manufacturing such that the shell comprises variable thickness, the variable thickness configured to provide a thicker region at areas of higher expected stress as the shell is loaded due to use of the shell by a patient wearing the shell to support the patient and a thinner region at areas of lower expected stress as the shell is loaded due to the use of the shell by the patient wearing the shell to support the patient.

Another implementation of the present disclosure is a method for manufacturing a lower extremity prosthetic socket, according to some embodiments. In some embodiments, the method includes using a digital scanner to capture either an anatomical structure of a patient's distal limb or an anatomical structure of a cast of the patient's distal limb to generate a scan file, converting the scan file to a design file, modifying the design file, and additively manufacturing the design file to produce the lower extremity prosthetic socket using an additive manufacturing device. The lower extremity prosthetic socket includes a shell having variable thickness along a longitudinal length of the shell and configured to directly couple, at an outer surface, with a pylon at a distal end. In some embodiments, the method also includes uploading the design file to the additive manufacturing device.

### BRIEF DESCRIPTION OF THE DRAWINGS

The disclosure will become more fully understood from the following detailed description, taken in conjunction with the accompanying figures, wherein like reference numerals refer to like elements, in which:

FIG. 1 is a front view of a prosthetic device, according to some embodiments.

FIG. 2 is a side view of the prosthetic device of FIG. 1, according to some embodiments.

FIG. 3 is a top view of the prosthetic device of FIG. 1, according to some embodiments.

FIG. 4 is a flow diagram of a process for manufacturing the prosthetic device of FIGS. 1-3, according to some embodiments.

FIG. 5 is a system for additive manufacturing that can be used to manufacture the prosthetic device of FIGS. 1-3, according to some embodiments.

### DETAILED DESCRIPTION

Before turning to the FIGURES, which illustrate the exemplary embodiments in detail, it should be understood that the present application is not limited to the details or methodology set forth in the description or illustrated in the FIGURES. It should also be understood that the terminology is for the purpose of description only and should not be regarded as limiting.

### Overview

Referring generally to the FIGURES, additive manufacturing is used to produce prosthetic and/or orthotic devices with variable wall thickness. The variable wall thickness facilitates improved fit and comfort, and can facilitate distribution of stresses. The prosthetic may be a lower extremity prosthetic socket. The prosthetic socket can be tailored in design for patients whose residual limb are either above-the-knee (AK) or below-the-knee (BK), according to some embodiments. The intended function of the prosthetic socket is to prove proper distribution of forces across a distal limb as well as provide stability when being worn and used by the patient.

The lower extremity prosthetic socket may have variable cross-sectional thickness. The thickness of the prosthetic socket can vary throughout based on the anatomy of the patient's residual limb as well as the requirements of the patient for use and functionality. The variable thickness can provide flexibility in areas of increased motion as well as provide increased structural support to areas of high stress.

The lower extremity prosthetic socket is produced using additive manufacturing, according to some embodiments. A method for creating the device includes taking either a three dimensional scan of a patient's residual limb or a three dimensional scan of a cast of a patient's residual limb, according to some embodiments. The scan is then converted to a computer assisted design (CAD)/computer assisted manufacturing (CAM) file, according to some embodiments. Build-ups, reductions, and other modifications are made to the CAD/CAM file to generate a 3D model of the device based on the anatomy and requirements of the patient, according to some embodiments. The CAD/CAM model of the device is then uploaded to an additive manufacturing machine, such as a 3D printer, where the device is then created layer by layer, according to some embodiments. The end result is a lower extremity prosthetic socket with variable thickness that conforms to the need and anatomy of the patient's distal limb, according to some embodiments.

The techniques described herein for additive manufacturing can additionally be used to manufacture the prosthetic, orthotic, connection insert, or related medical devices as described in U.S. Patent Application Pub. No.: 2018/0353308 A1, filed Jul. 31, 2018, the entire disclosure of which is incorporated by reference herein. Further, any of the additive manufacturing techniques as described in U.S. Patent Application Pub. No.: 2018/0353308 A1 may be used to manufacture any of the devices described herein.

In some embodiments, the prosthetic, orthotic, connection insert, protective device, etc., as described herein are manufactured using any of the techniques as described in U.S. Pat. No. 10,766,246 B2, filed Dec. 15, 2014, the entire disclosure of which is incorporated by reference herein.

The present disclosure can also include systems and methods for creating custom-fit prosthetic devices, orthotic devices, and related medical devices via three-dimensional printing (3D printing) or additive manufacturing techniques are described. For example, the described systems and methods may be used to fabricate prosthetic devices, cranial remolding orthosis devices, ankle-foot orthosis devices, upper and lower extremity prosthetic devices, shoe inserts, and the like. Through the described systems and methods, a residual limb or other body part of a patient is scanned and analyzed to determine measurements and characteristics of the residual limb. The measurements and characteristics of the residual limb are used to design a customized device for the residual limb. The customized device uses multiple different materials. For example, the customized device may use a first material for a frame and a second material for a liner, wherein the first material is more rigid than the second material. The customized device is fabricated using a three-dimensional printer that is capable of printing and bonding multiple different materials at the same time.

### Lower Extremity Prosthetic Socket

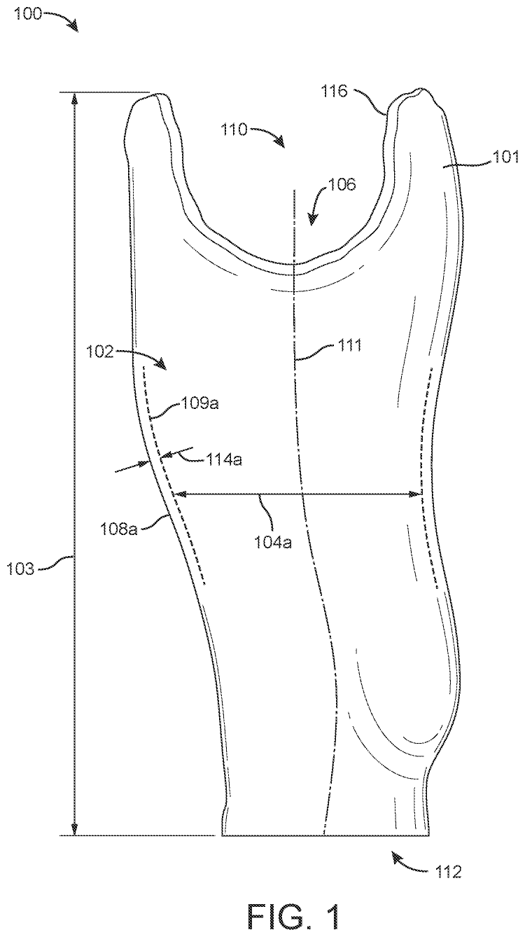

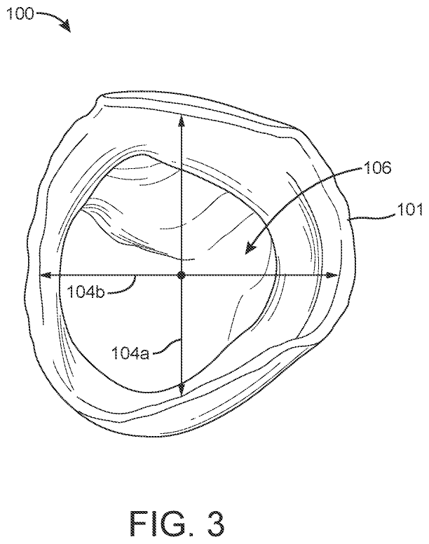

Referring particularly to FIGS. 1-3, a prosthetic, a prosthetic device, a lower extremity prosthetic socket, a prosthetic socket, etc., shown as prosthetic socket 100 is shown, according to an exemplary embodiment. Prosthetic socket 100 is configured for use with a residual limb (e.g., a lower limb, a leg limb, etc.) of a patient or user. Prosthetic socket 100 includes a shell, a structural member, a sidewall, etc., shown as shell 101. Prosthetic socket 100 (or more particularly shell 101) can be manufactured using a 3d printing or an additive manufacturing technique.

Prosthetic socket 100 can be configured for use with patients whose residual limbs are either above-the-knee (AK) or below-the-knee (BK). Prosthetic socket 100 can be placed or installed onto the patient's residual limb to provide proper distribution of forces across the residual limb (e.g., a distal limb) and to provide stability for the residual limb when the prosthetic socket 100 is worn and used (e.g., when the user ambulates while wearing prosthetic socket 100). Shell 101 can include an inner volume 106 (e.g., a void, a cavity, etc.) configured to receive the patient's residual limb. The patient may insert their residual limb into the inner volume 106 of shell 101 at an upper or proximate end 110 of shell 101. In some embodiments, a geometry of shell 101 (e.g., a shape of inner volume 106) corresponds to or matches a shape or geometry of the patient's residual limb. The shell 101 can be configured to surround, enclose, or fully receive the patient's residual limb. In some embodiments, structural contours (e.g., first exterior contour 108a, second exterior contour 108b, etc.) on either an exterior surface or an interior surface of shell 101 or a general shape of shell 101 match or correspond with anatomical contours of the patient's residual limb.

Prosthetic socket 100 (e.g., shell 101 of prosthetic socket 100) is configured to provide proper distribution of forces across the patient's residual or distal limb as well as across shell 101 when prosthetic socket 100 is in use (e.g., when the patient wears prosthetic socket 100 and walks or otherwise loads shell 101 in tension, compression, torsion, etc.).

In some embodiments, shell 101 is manufactured or produced from a material such as a thermoplastic (e.g., a versatile thermoplastic such as nylon). A material composition of the material of the socket 100 or shell 101 can be lightweight or have suitable density for improved patient comfortability. In some embodiments, the material of shell 101 is also selected or configured so that shell 101 can undergo flexion, twisting, etc., or otherwise experience deformation when being loaded or used by the patient. In some embodiments, the material composition facilitates variable flexibility and rigidity throughout shell 101 (e.g., along a height of shell 101 from the proximate end 110 to a distal or second end 112 that is opposite the proximate end 110). A longitudinal axis, a central axis, or a dimension can be defined between the proximate end 110 and the distal end 112. For purposes of illustration, FIG. 1 includes a centerline 111 extending through shell 101.

In some embodiments, the material composition of shell 101 facilitates minor adjustments to be made to an overall shape of shell 101 by heating shell 101. Shell 101 can be heated in particular areas where a plastic deformation is desired, deformed (e.g., by a manufacturer) and cooled so that the deformation remains. In this way, shell 101 can be adjusted or deformed plastically (or elastically, without heat addition) without sustaining structural damage. For example, shell 101 can be modified (e.g., by adding heat and applying a force) to reduce in overall height 103 (measured from proximate end 110 to distal end 112 along the longitudinal direction or along the dimension).

For example, shell 101 may include one or more dimensions 102 such as a first width 104a of inner volume 106, a first thickness 114a, a second width 104b of inner volume 106, a second thickness 114b, various circumferences, etc. By providing heat and applying forces or moments to shell 101, one or more of the dimensions 102 can be adjusted. For example, a curvature of shell 101 at a base of shell 101 of prosthetic socket 100 (e.g., at distal end 112) can be adjusted by applying heat and plastically deforming shell 101.

Referring particularly to FIG. 1, shell 101 includes first exterior contour 108a and a first interior contour 109a. First exterior contour 108a extends along the exterior surface of shell 101, while first interior contour 109a extends along the interior surface of shell 101 for the front view as shown in FIG. 1. A thickness 114a of shell 101 is defined between the first exterior contour 108a and the first interior contour 109a as shown in the front view of shell 101.

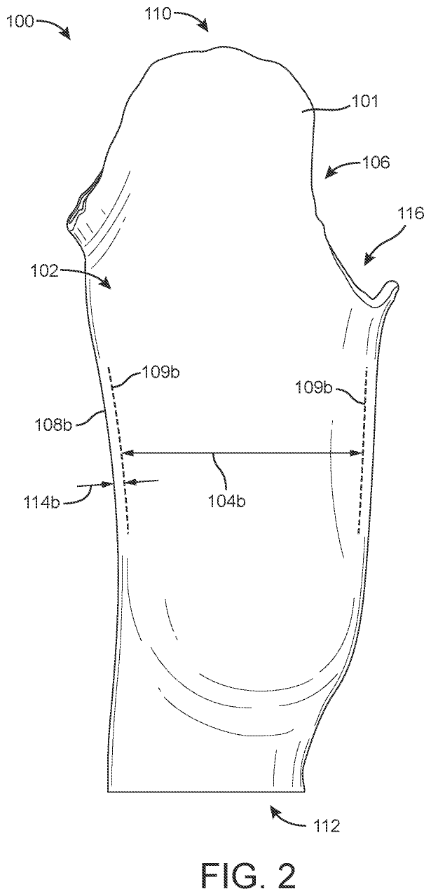

Referring particularly to FIG. 2, shell 101 includes a second exterior contour 108b and a second interior contour 109b. Second exterior contour 108b extends along the exterior surface of shell 101, while second interior contour 109b extends along the interior surface of shell 101 for the side view as shown in FIG. 1. A thickness 114b of shell 101 is defined between the second exterior contour 108b and the second interior contour 109b. It should be understood that thickness 114b and thickness 114a both show the thickness of shell 101 but at different orientations and different positions along the height of shell 101. Thickness 114a and thickness 114b for a same location along the height of shell 101 may be the same (e.g., uniform) or may be different. It should be understood that any number of thicknesses of shell 101 can be defined taken from any orientation of shell 101 (e.g., at any view, at a view 45 degrees between the front view and the side view, etc.).

Referring to FIGS. 1-3, in some embodiments, the thickness (e.g., thickness 114a and/or thickness 114b, etc.) of shell 101 is constant along the height of shell 101. In some embodiments, the thickness of shell 101 is non-constant along the height of shell 101 and is instead variable. For example, the thickness of shell 101 may be greatest at the distal end 112 of shell 101 and decrease to a lowest value as the proximate end 110 of shell 101. It should also be understood that the thickness of shell 101 may vary at different orientations or angles relative to centerline 111 or a longitudinal axis extending through shell 101. In this way, different areas or portions of shell 101 (e.g., different locations along the height of shell 101, or along centerline 111, or along the longitudinal axis, etc.) can have different thicknesses. The different thicknesses can correspond to an amount of deformation (e.g., plastic or elastic) or flexion (e.g., plastic or elastic) that the shell 101 experiences (during use of the prosthetic socket 100 or when heat is applied to adjust the geometry of shell 101). In some embodiments, areas where shell 101 is thinner (e.g., the thickness is at a decreased value) experience greater degrees or amounts of deformation or flexion. Similarly, areas where shell 101 is thicker (e.g., the thickness is at an increased value) experience a smaller degree or amount of deformation or flexion relative to the thinner areas, according to some embodiments. In some embodiments, the thickness of shell 101 (e.g., thickness 114a and/or thickness 114b) is designed or configured to provide desired flexion or deformation when used by the patient to improve stability and/or comfort of the prosthetic socket 100.

Referring to FIGS. 1-3, shell 101 is shown to include one or more trimlines 116 at proximate end 110 of the prosthetic socket 100. Shell 101 may taper (e.g., decreasing thickness) at trimlines 116. In some embodiments, the trimlines 116 can be adjusted (e.g., by applying heat and plastically deforming shell 101, or during the manufacturing/design process of shell 101) to fit the requirements of the patient's residual limb.

In some embodiments, prosthetic socket 100 is configured to surround and attach to the patient's residual limb. In some embodiments, prosthetic socket 100 is configured to interface with a prosthetic leg (e.g., a pylon and an artificial foot or leg) at distal end 112. Prosthetic socket 100 thereby provides stability across the patient's distal limb and provides a proper distribution of forces when in use with the prosthetic leg. Shell 101, or more generally, prosthetic socket 100, can be fabricated or manufactured using additive manufacturing as described in greater detail herein. In some embodiments, shell 101 is a single unit that is additively manufactured with a single material.

The shell 101 has thickness 114 (e.g., thickness 114a and/or thickness 114b) that may transition between the first thickness 114a and the second thickness 114b at different spatial locations along the shell 101. The thickness 114 of the shell 101 may be uniform or may vary spatially at different positions. For example, areas of the shell 101 that are anticipated or expected to undergo higher stress may have an increased thickness relative to other areas that are expected to undergo lower stress during use of the prosthetic socket 100 (or vice versa). In some embodiments, different areas of the shell 101 that should deform to a shape of the user's residual limb have a decreased thickness to facilitate controlled flexing or bending of the shell 101 to facilitate comfort and proper fit of the shell 101. In some embodiments, the thickness of the shell 101 increases from one end to another end of the shell 101 so that the thickness of the shell 101 proximate the one end is greater than thickness of the shell 101 at the other end. In some embodiments, variation of the thickness of the shell 101 is configured based on patient activity level, weight, etc.

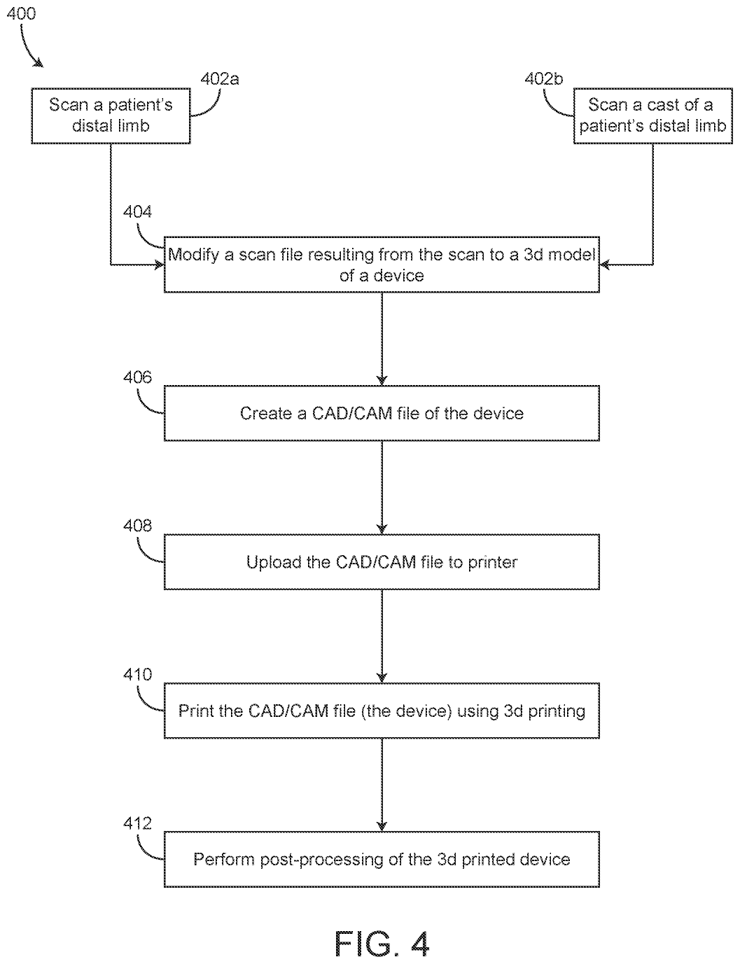

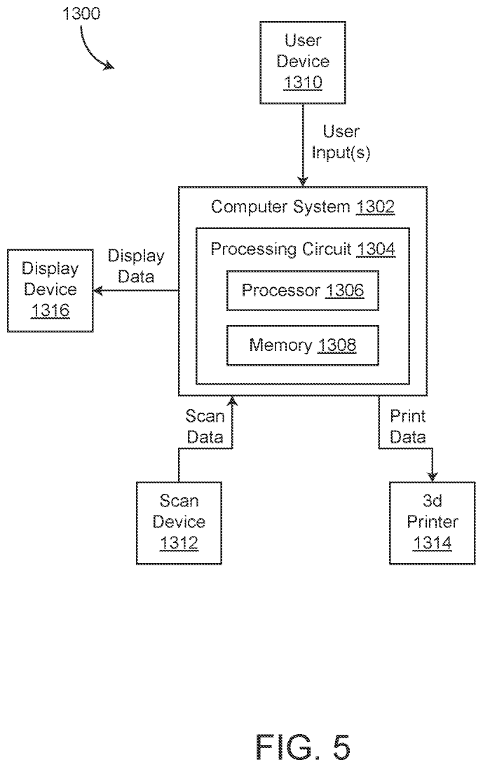

Referring particularly to FIG. 4, a flow diagram of a process 400 for producing or manufacturing the prosthetic socket 100 of FIGS. 1-3 is shown, according to some embodiments. Process 400 includes steps 402-412 and can be performed using an additive manufacturing system (e.g., system 1300 as described in greater detail below with reference to FIG. 5).

Process 400 includes scanning a patient's distal or residual limb (step 402a) or scanning a cast of a patient's distal limb (step 402b). In some embodiments, step 402a or step 402b is performed using a scanning device (e.g., scan device 1312 as described in greater detail below with reference to FIG. 5). The patient's limb can be scanned directly (step 402a), or a cast of the patient's limb may be scanned (step 402b). In some embodiments, performing step 402a or step 402b results in the generation of a scan file.

Process 400 includes modifying a scan file resulting from the scan (e.g., resulting from performing step 402a or step 402b) to a 3d model of a device (e.g., the prosthetic socket 100) (step 404), according to some embodiments. In some embodiments, step 404 is performed on a computer system based on one or more user inputs or inputs from a health care provider. For example, step 404 can include adjusting a thickness of the device of the scan file at different locations. In some embodiments, step 404 includes digitally using buildups or reductions to the thickness of the 3d model of the device to achieve a desired thickness that yields a desired corresponding deformation or flexion when the device is loaded. For example, step 404 can be performed by computer system 1302 based on one or more user inputs or inputs from a health care provider obtained from user device 1310 (described in greater detail below with reference to FIG. 5).

Process 400 includes creating a computer assisted design (CAD) and/or a computer assisted manufacturing (CAM) file of the device (e.g., the prosthetic socket 100) (step 406), according to some embodiments. Process 400 also includes uploading the CAD/CAM file to a printer (e.g., 3d printer 1314) (step 408), according to some embodiments. Steps 406 and 408 can be performed by computer system 1302 (e.g., in response to a user input such as from a health care provider) as described in greater detail below with reference to FIG. 5.

Process 400 includes printing the CAD/CAM file using 3d printing (e.g., to generate the device, the prosthetic socket 100, etc.) (step 410), according to some embodiments. In some embodiments, step 410 includes performing additive manufacturing (e.g., dispensing or outputting layers consecutively on top of each other) to produce the device. In some embodiments, the additive manufacturing is performed using a single uniform material such as a thermoplastic (e.g., nylon). The resulting device or 3d printed component can have variable thickness as defined by the CAD/CAM file.

Process 400 includes performing post-processing on the 3d printed device (step 412), according to some embodiments. For example, step 412 can include removing excess material that is dispensed during step 410 (e.g., during fabrication of the device). Step 412 can be performed by a technician. Additional post-processing can be performed based on anatomy or needs of the patient.

In some embodiments, the device that is produced by performing process 400 is a lower extremity prosthetic socket, with a varying thickness (e.g., cross-sectional thickness) throughout. The device can provides proper stability and distribution of forces when worn, and is produced using additive manufacturing techniques. The thickness of the device can be modified in any area to accommodate the anatomy of the patient as well as any additional requirements the patient may have. The device is created using 3D printing, wherein the material composition is of a single uniform substance and can provide extra comfort to the patient when worn due to its lightweight properties, according to some embodiments.

### Additive Manufacturing System Architecture

Referring now to FIG. 5, a system 1300 for additive manufacturing of prosthetic, orthotic, or protective devices is shown, according to some embodiments. System 1300 includes a user device 1310, a display device 1316, a computer system 1302, a scan device 1312, and a 3d printer or additive manufacturing machine 1314.

Computer system 1302 is configured to receive scan data from scan device 1312, according to some embodiments. Computer system 1302 can be a desktop computer, a laptop, a remote computing system, a smart phone, a tablet, a personal computing device, etc. Computer system 1302 includes a processing circuit 1304 having memory 1308 and a processor 1306. Processor 1306 can be implemented as a general-purpose processor, an application specific integrated circuit (ASIC), one or more field programmable gate arrays (FPGAs), a group of processing components, or other suitable electronic processing components.

Memory 1308 (e.g., memory, memory unit, storage device, etc.) may include one or more devices (e.g., RAM, ROM, Flash memory, hard disk storage, etc.) for storing data and/or computer code for completing or facilitating the various processes, layers and modules described in the present application. Memory 1308 may be or include volatile memory or non-volatile memory. Memory 1308 may include database components, object code components, script components, or any other type of information structure for supporting the various activities and information structures described in the present application. According to an exemplary embodiment, memory 1308 is communicably connected to processor 1306 via processing circuit 1304 and includes computer code for executing (e.g., by processing circuit 1304 and/or processor 1306) one or more processes described herein.

Computer system 1302 can be configured to run CAD computer software to facilitate the design and production of any of prosthetic socket 100, orthotic device 200, and/or protective device 300. Computer system 1302 is configured to receive scan data from scan device 1312, according to some embodiments. In some embodiments, the scan data is a scan file obtained from scan device 1312. In some embodiments, a technician may scan device 1312 to scan a patient's residual limb or a cast of the patient's residual limb, thereby generating the scan data.

When the scan data is provided to computer system 1302, computer system 1302 can generate a CAD or CAM file. A user (e.g., a health care provider) can then provide inputs (e.g., via user device 1310) to adjust geometry, thickness, etc., of the CAD or CAM file. More generally, computer system 1302 may use the scan data to generate a digital representation of a device to be manufactured for the patient's residual limb. Computer system 1302 can provide display data to display device 1316 (e.g., a computer screen, a display screen, etc.) so that the digital representation is visually displayed in real-time. The user or health care provider can then view real-time changes or updates as the user changes or adjusts the CAD or CAM file.

For example, the user may adjust the CAD or the CAM file so that the design gradually tapers or thickens in different areas. In some embodiments, the user or the health care provider may use data from different experiments to identify areas where a patient may experience high stress. The user may decrease thickness of the CAD or CAM file at areas where high stress is experienced so that the 3d printed device may flex or deform. This can allow the 3d printed device to be more comfortable for the patient. In some embodiments, thickness of the 3d printed devices is maintained above a minimum thickness value. The user can also use knowledge regarding different weight lines of the patient to determine which areas of the CAD or CAM file/model should have decreased or increased thickness. The user may also use historical data to determine which areas or portions of the 3d printed device or the CAD/CAM file/model should have increased or decreased thickness (e.g., wall thickness).

Once the user (e.g., the health care provider) has adjusted or manipulated the CAD/CAM file/model, the user can prompt computer system 1302 to export the file/model to 3d printer 1314 as print data. Computer system 1302 can convert the adjusted, manipulated, or updated CAD/CAM file/model to a file type that is compatible with 3d printer 1314 (e.g., a Standard Tessellation Language (STL) file). Computer system 1302 then provides the print data to 3d printer 1314.

The 3d printer 1314 can be any additive manufacturing machine or device that is configured to successively provide or discharge layers of material onto each other to form or construct a part. 3d printer 1314 may be configured to dispense material (e.g., one or more powder materials that can form nylon when combined with fusing/detailing agents and exposed to fusing light, or any other dispensable materials) in layers to fabricate the CAD/CAM file.

Advantageously, the systems and methods described herein can be used to produce 3d printed prosthetics, orthotics, or protective devices. Traditional molding methods do not offer the same flexibility of variable wall thickness as does additive manufacturing. The variable wall thickness is achieved using additive manufacturing (e.g., 3d printing) and can facilitate improved fit, comfort, and stress distribution.

### Configuration of Exemplary Embodiments

As utilized herein, the terms “approximately”, “about”, “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claim.

It should be noted that the terms “exemplary” and “example” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like, as used herein, mean the joining of two members directly or indirectly to one another. Such joining may be stationary (e.g., permanent, etc.) or moveable (e.g., removable, releasable, etc.). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” “between,” etc.) are merely used to describe the orientation of various elements in the figures. It should be noted that the orientation of various elements may differ according to other exemplary embodiments, and that such variations are intended to be encompassed by the present disclosure.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

It is important to note that the construction and arrangement of the systems as shown in the exemplary embodiments is illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the components described herein may be constructed from any of a wide variety of materials that provide sufficient strength or durability, in any of a wide variety of colors, textures, and combinations. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claim.

What is claimed is:

1. A method comprising:
obtaining scan data of a patient's lower limb;
using computer assisted design to generate a print file of an ankle foot orthotic (AFO) based on the scan data; and
performing additive manufacturing by a 3D printer to produce the AFO using the print file of the AFO.

2. The method of claim 1, wherein the AFO comprises an outer shell configured to receive a patient interfacing insert, wherein at least one of the outer shell or the patient interfacing insert are manufactured by the 3D printer.

3. The method of claim 2, wherein using the computer assisted design further comprises designing a print file of the patient interfacing insert based on the scan data, and the method further comprises performing additive manufacturing by the 3D printer to produce the patient interfacing insert of the AFO using the print file of the patient interfacing insert.

4. The method of claim 1, wherein the AFO comprises an outer shell, the outer shell comprising a plurality of mounting structures for at least one strap.

5. The method of claim 1, wherein using the computer assisted design to generate the print file comprises modifying a position, thickness, and trim area of the print file such that the AFO produced using the print file is variable along the patient's calf, ankle, and foot.

6. The method of claim 1, wherein using the computer assisted design to generate the print file includes drawing lines and shapes onto the scan data of the patient's lower limb and generating the print file based on the lines and shapes.

7. The method of claim 1, wherein the AFO has the form of a wearable device configured to be worn on the patient's lower limb and at least partially surround a portion of the patient's calf, ankle, or foot.

8. A method for manufacturing a lower extremity prosthetic socket, the method comprising:
using a digital scanner to capture either an anatomical structure of a patient's distal limb or an anatomical structure of a cast of the patient's distal limb to generate a scan file;
converting the scan file to a design file;
modifying the design file; and
additively manufacturing the design file to produce the lower extremity prosthetic socket using an additive manufacturing device such that the lower extremity prosthetic socket has variable thickness targeting areas of expected stress.

9. The method of claim 8, wherein modifying the design file comprises using build-ups or reductions to a thickness of a shell of the design file.

10. The method of claim 8, wherein the lower extremity prosthetic socket comprises variable thickness along a dimension of the lower extremity prosthetic socket.

11. The method of claim 10, wherein the variable thickness is configured to accommodate the anatomical structure of the patient's distal limb.

12. The method of claim 11, wherein the variable thickness targets areas of high stress and distributes stress throughout the lower extremity prosthetic socket.

13. The method of claim 8, further comprising identifying areas of high stress based on patient data and modifying the design file such that the lower extremity prosthetic socket has variable thickness targeting the areas of high stress and such that the lower extremity prosthetic socket distributes stress throughout the lower extremity prosthetic socket.

14. The method of claim 8, wherein the lower extremity prosthetic socket is configured to distribute forces.

15. The method of claim 8, comprising modifying the design file and additively manufacturing the design file such that the lower extremity prosthetic socket comprises trimlines at a proximate end, the trimlines being tapered and having thickness less than the thickness of the lower extremity prosthetic socket proximate a distal end such that the lower extremity prosthetic socket conforms to the anatomical structure of the patient's distal limb.

16. The method of claim 8, comprising modifying the design file and additively manufacturing the design file such that the lower extremity prosthetic socket is configured to directly couple, at an outer surface, with a pylon at a distal end.

17. The method of claim 8, further comprising plastically or elastically deforming a part of the lower extremity prosthetic socket after producing the lower extremity prosthetic socket, wherein the part of the lower extremity prosthetic socket is configured to undergo deformation without sustaining structural damage.

18. The method of claim 17, comprising modifying the design file and additively manufacturing the design file such that an amount of the deformation or flexion at a particular position along the lower extremity prosthetic socket is inversely proportional to a thickness at the particular position along the lower extremity prosthetic socket.

19. The method of claim 8, wherein the design file is at least one of a computer assisted design (CAD) file or a computer assisted manufacturing (CAM) file.

20. The method of claim 8, wherein the additive manufacturing device is a 3d printer configured to output layers of material on top of each other in succession to produce the lower extremity prosthetic socket.
